(19) United States
(12) Patent Application Publication
(10) Pub. No.: US 2010/0306868 A1
(43) Pub. Date: Dec. 2, 2010

(54) QUADRUPLE TRANSGENIC NON-HUMAN ANIMAL

(76) Inventors: Bernd Bohrmann, Riehen (CH); Antonio Iglesias, Freiburg (DE); Hansruedi Loetscher, Moehlin (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/740,112

(22) PCT Filed: Oct. 23, 2008
(86) PCT No.: PCT/EP08/08971

§ 371 Date: Apr. 28, 2010

(30) Foreign Application Priority Data
Nov. 1, 2007 (EP) 07119837.8

(51) Int. Cl. A01K 67/027; C12N 5/07; C12N 15/89

(52) U.S. Cl. 800/18; 800/13; 435/325; 800/25; 435/1.1

(57) ABSTRACT

The present invention relates to a transgenic non-human animal whose genome comprises a) a first transgenic DNA sequence encoding a human APP Swedish or a human APP London protein, wherein said first transgenic DNA sequence is operably linked to a first promoter; b) a second transgenic DNA sequence encoding a human Presenilin 2 protein comprising a N141I substitution, wherein said second transgenic DNA sequence is operably linked to a second promoter; c) a third transgenic DNA sequence encoding a light chain of a antibody directed against the amyloid peptide, wherein said third transgenic DNA sequence is operably linked to a third promoter and; d) a forth transgenic DNA sequence encoding a heavy chain of said antibody, wherein said forth transgenic is operably linked to the third promoter or to a forth promoter, and methods for producing said animal.

Alzheimer's disease (AD) is a neurodegenerative disease which is characterized by memory loss and decline of cognitive functions. The prevalence of AD approximately doubles with every five years over the age of 65. Overall, approximately 5 to 10% of those over 65 are affected. The clinical diagnosis of AD remains an exclusion diagnosis. Only post-mortem microscopic examination of the brain offers a definitive diagnosis based on the presence of extracellular plaques containing fibrils of amyloid-beta (A-beta) peptide and intracellular tangles containing polymerized phosphorylated Tau protein. In most of the cases, AD occurs late in onset and sporadically. The early onset familial AD represents a minority of the cases, but they have been extremely important for the progression in the understanding of the disease mechanisms. Known mutations are the Swedish Mutation (SFAD) which affect codons 670/671 (K670+N and M671+L) and the London mutation which affects codon 717 of APP. All these mutations affect the proteolytic processing of APP yielding more amyloidogenic peptides.

APP can be processed by at least 3 secretases: alpha-, beta-, and gamma-secretases. Beta-secretase initiates A-beta peptide generation by cleaving APP after Methionine 671 (APP770 numbering) leading to a 12 kd retained membrane carboxyterminal fragment. The 12 kd fragment may then undergo gamma-secretase cleavage within the hydrophobic transmembrane domain to release the 40, 42, or 43 residue A-beta peptides (Seubert P, Vigo-Pelfrey C, Esch F, Lee M, Dovey H, Davis D, Sinha S, Schlossmacher M, Whaley J, Swindlehurst C. Isolation and quantification of soluble Alzheimer's beta-peptide from biological fluids. Nature. 1992. 359: 325-7).

Current treatments for AD provide only modest symptomatic relief. There is a high unmet medical need for disease modifying agents that slow the course of the disease and prevent or delay the disease in susceptible individuals. The development of such agents requires, among others, progress in the understanding of the molecular basis of the disease and in the development of animal models.

A number of transgenic lines overexpressing proteins relating with AD are known, for example double transgenic mice overexpressing APP London and beta secretase.

The present invention provides a transgenic non-human animal whose genome comprises a) a first transgenic DNA sequence encoding a human APP protein comprising a mutation associated with Alzheimer's disease (AD) or AD-type pathology, wherein said first transgenic DNA sequence is operably linked to a first promoter;

b) a second transgenic DNA sequence encoding a human Presenilin 2 protein comprising a mutation associated with Alzheimer's disease (AD) or AD-type pathology, wherein said second transgenic DNA sequence is operably linked to a second promoter;
c) a third transgenic DNA sequence encoding a light chain of a human antibody directed against the amyloid peptide, wherein said third transgenic DNA sequence is operably linked to a third promoter and;
a forth transgenic DNA sequence encoding a heavy chain of said human antibody, wherein said forth transgenic is operably linked to a forth promoter or to the third promoter.

The term “Alzheimer's disease (AD) pathology” refers to pathological changes in AD patients such as for example loss of neuronal synaptic density and synapse number, decreased cognitive ability, and memory loss. They also include diffuse and neuritic plaques in the brain, which are predominantly composed of amyloid beta peptide, neuronal and/or glial inclusions or insoluble deposits that stain positively with anti-Abeta antibodies.

In particular, the present invention provides a transgenic non-human animal whose genome comprises

a) a first transgenic DNA sequence encoding a human APP Swedish or a human APP London protein, wherein said first transgenic DNA sequence is operably linked to a first promoter;
b) a second transgenic DNA sequence encoding a human Presenilin 2 protein comprising a N141I substitution, wherein said second transgenic DNA sequence is operably linked to a second promoter;
c) a third transgenic DNA sequence encoding a light chain of a human antibody directed against the human amyloid peptide, wherein said third transgenic DNA sequence is operably linked to a third promoter and;
d) a forth transgenic DNA sequence encoding a heavy chain of said human antibody, wherein said forth transgenic is operably linked to the third promoter or to a forth promoter.

The present invention also relates to descendants of the transgenic non-human animals as provided by the invention, obtained by breeding with the same or with another genotype. Preferably, the descendant is obtained by breeding with the same genotype. Said descendant comprises the same transgenic DNA sequences as the quadruple non-human animal described above.

Furthermore, the present invention relates to a cell line or primary cell culture derived from a quadruple non-human transgenic animal or its descendants as described above.

In addition, the present invention also provides a tissue or an organ explant or culture thereof, derived from a non-human transgenic animal or its descendants as described above.

The present invention also provides a tissue or cell extract derived a non-human transgenic animal or its descendants as described above.

The present invention also provides a method for generating a transgenic non-human animal whose genome comprises transgenic DNA encoding a human APP Swedish or APP London, a human Presenilin 2 protein comprising N141I substitution, and a heavy and a light chain of a human antibody directed against the beta amyloid peptide comprising

a) Co-injecting a genetic construct comprising said first transgenic DNA sequence encoding a human APP Swedish or APP London, and a genetic construct comprising the second transgenic DNA sequence encoding a human Presenilin 2 protein with a N141I substitution into a non-human zygote or an non-human embryonic stem cell, and generating a double transgenic non-human animal from said zygote or embryonic stem cell;
b) Co-injecting a genetic construct comprising the third transgenic DNA sequence encoding a light chain of a human antibody directed against the amyloid peptide, and a genetic construct comprising the forth transgenic DNA sequence encoding a heavy chain of said human antibody into a non-human zygote or a non-human embryonic stem cell, and generating a double transgenic non-human animal from said zygote or embryonic stem cell,
c) Mating transgenic animals of a) and b) and thereby producing a quadruple transgenic non-human animal whose genome comprises transgenic DNA encoding, a human APP Swedish or APP London, a human Presenilin 2 protein comprising a N141I substitution, a heavy and a light chain of a human antibody directed against the beta amyloid peptide.

Alternatively, in step c) of the above mentioned methods the third and forth transgenic DNA sequences may be on the same construct.

The present invention also provides a method for generating a transgenic non-human animal expressing a human APP Swedish or APP London, a human Presenilin 2 protein comprising N141I substitution and a heavy and a light chain of a human antibody directed against the beta amyloid peptide comprising

a) Co-injecting a genetic construct comprising said first transgenic DNA sequence encoding a human APP Swedish or APP London wherein said first transgenic DNA sequence is operably linked to a first promoter, and a genetic construct comprising the second transgenic DNA sequence encoding a human Presenilin 2 protein with a N141I substitution into a non-human zygote or an non-human embryonic stem cell, wherein said second transgenic DNA sequence is operably linked to a second promoter, and generating a double transgenic non-human animal from said zygote or embryonic stem cell;
b) Co-injecting a genetic construct comprising the third transgenic DNA sequence encoding a light chain of a human antibody directed against the human amyloid peptide, wherein said third transgenic DNA sequence is operably linked to a third promoter and a genetic construct comprising the forth transgenic DNA sequence encoding a heavy chain of said human antibody into a non-human zygote or a non-human embryonic stem cell, wherein said forth transgenic DNA sequences is operably linked to forth promoter, and generating a double transgenic non-human animal from said zygote or embryonic stem cell,
c) Mating transgenic animals of a) and b) and thereby producing a quadruple transgenic non-human animal expresses a human APP Swedish or APP London, a human Presenilin 2 protein comprising a N141I substitution, and a heavy and a light chain of a human antibody directed against the beta amyloid peptide.

Alternatively, in step c) of the above mentioned methods the third and forth transgenic DNA sequences may be on the same construct operably linked to the third promoter.

The present invention further provides a quadruple transgenic non-human animal produced by the method described above.

APP

APP means amyloid precursor protein. A number of cDNA forms of APP have been identified, encoding among others the three most abundant isoforms APP695, APP751, and APP770. These forms arise from a single precursor RNA by alternate splicing. The gene spans more than 175 kb with 18 exons (Yoshikai S, Sasaki H, Doh-ura K, Furuya H, Sakaki Y. Genomic organization of the human amyloid beta-protein precursor gene. Gene. 1990. 87(2):257-63). APP contains an extracellular domain, a transmembrane region and a cytoplasmic domain. A-beta consists of up to 28 amino acids just outside the hydrophobic transmembrane domain and up to 15 residues of this transmembrane domain. Thus, A-beta is a cleavage product derived from APP which is normally found in brain and other tissues such as heart, kidney and spleen. However, A-beta deposits are usually found in abundance only in the brain. The larger alternate forms of APP (APP751, APP770) consist of APP695 plus one or two additional domains. APP751 consists of all 695 amino acids of APP695 plus an additional 56 amino acids which has homology to the Kunitz family of serine protease inhibitors (KPI) (Tanzi R E, McClatchey A I, Lamperti E D, Villa-Komaroff L, Gusella J F, Neve R L. Protease inhibitor domain encoded by an amyloid protein precursor mRNA associated with Alzheimer's disease. Nature. 1988. 331:528-30; Weidemann A, Konig G, Bunke D, Fischer P, Salbaum J M, Masters C L, Beyreuther K Identification, biogenesis, and localization of precursors of Alzheimer's disease A4 amyloid protein. Cell. 1989 Apr. 7; 57(1):115-26. Kitaguchi N, Takahashi Y, Tokushima Y, Shiojiri S, Ito H Novel precursor of Alzheimer's disease amyloid protein shows protease inhibitory activity. Nature. 1988. Feb. 11; 331(6156): 530-2; Tanzi R E, St George-Hyslop P H, Haines J L, Polinsky R J, Nee L, Foncin J F, Neve R L, McClatchey A I, Conneally P M, Gusella J F. The genetic defect in familial Alzheimer's disease is not tightly linked to the amyloid beta protein gene. Nature. 1987. 329 (6135):156-7). APP770 contains all 751 amino acids of APP751 and an additional 19 amino acid domain homologous to the neuron cell surface antigen OX-2 (Weidemann et al. 1989; Kitaguchi et al. 1988). Unless otherwise noted, the amino acid positions referred to herein are the positions as they appear in APP770. The amino acid number of equivalent positions in APP695 and APP751 differ in some cases due to the absence of the OX-2 and KPI domains. By convention, the amino acid positions of all forms of APP are referenced by the equivalent positions in the APP770 form. Unless otherwise noted, this convention is followed herein. Unless otherwise noted, all forms of APP and fragments of APP, including all forms of A-beta, referred to herein are based on the human APP amino acid sequence. APP is post-translationally modified by the removal of the leader sequence and by the addition of sulfate and sugar groups. The term “APP” includes also mutant APP, such for example as APPswedish and APP London.

A human APP protein comprising a mutation associated with Alzheimer's disease (AD) or AD-type pathology is preferably APPswedish and APPlondon.

The term “APP Swedish” as used herein refers an APP isoform as defined above comprising a K670N/M671L substitution. Preferably the human APP Swedish is encoded by SEQ. ID NO: 1.

The term “APP London” as used herein refers to an APP isoform as defined above containing one or more of the either natural or artificial mutations which affect the production of amyloid peptides towards a specific increased production of A-beta 42 peptide. Preferred are natural APP mutations around the gamma-secretase cleavage site with pathological relevance including
T714I (Kumar-Singh S et al. Hum Mol Genet. 2000 Nov. 1; 9(18):2589-98.)
V715M (Ancolio K, et al., Proc Natl Acad Sci USA. 1999 Mar. 30; 96(7):4119-24.)
1716V (Eckman C B, et al., Hum Mol Genet. 1997 November; 6(12):2087-9.)
V717F (Murrell J, et al, Science. 1991 Oct. 4; 254(5028):97-9.)
V717G (Chartier-Harlin M C, et al., Nature. 1991 Oct. 31; 353(6347):844-6.)
V717I (Goate A, et al., Nature. 1991 Feb. 21; 349(6311):704-6)
V717L (Murrell J R, et al., Arch Neurol. 2000 June; 57(6):885-7.)
L723P (Kwok J B, et al., Ann Neurol. 2000 February; 47(2):249-53.)

Preferred are also artificial mutations (replacement of all residues in the transmembrane region outside of the A-beta domain) examples of which and their consequence on the gamma-secretase cleavage have been described by Lichtenthaler and co-workers (Lichtenthaler S F, et al., Proc Natl Acad Sci USA. 1999 Mar. 16; 96(6):3053-8).

More preferably, human APP London is human APP London containing the natural V717F mutation. Most preferably the human APP London is encoded by SEQ ID NO: 3.

Preferably, the first transgenic DNA sequence encodes human APPswedish.

Presenilin 2 (PS2)

Presenilin 2 (PS) is a protein of the gamma-secretase complex. The gamma-secretase is involved in the processing of the APP (see FIG. 1).

The term “DNA sequence encoding Presenilin 2” or “PS2 gene” as used herein refers to the mammalian gene first disclosed and described in U.S. application Ser. No. 08/496,841, filed on Jun. 28, 1995, and later described in Rogaev et al. (Nature. 1995; 376(6543):775-8) and Levy Lahad et al. (Ann Neurol. 1995; 38(4):678-80), including any allelic variants and heterospecific mammalian homologues. One human presenilin-2 (hPS2) cDNA sequence is disclosed herein as SEQ ID NO: 9.

The term “presenilin-2 gene” or “PS2 gene” primarily relates to a coding sequence, but can also include some or all of the flanking regulatory regions and/or introns. The term PS2 gene specifically includes artificial or recombinant genes created from cDNA or genomic DNA, including recombinant genes based upon splice variants. The presenilin-2 gene has also been referred to as the E5-1 gene.

A human Presenilin 2 protein comprising a mutation associated with Alzheimer's disease (AD) or AD-type pathology is preferably a human PS2 with N141I, T122P, M239V or M239I substitution (Shen et al., PNAS, 2007, 104(2):403-409). More preferable, the human Presenilin 2 protein comprising a mutation associated with Alzheimer's disease (AD) or AD-type pathology is a human PS2 with N141I substitution.

Therefore, the second transgenic DNA sequence preferably encodes a human PS2 with a N141I substitution. Most preferred is the DNA sequence encoding SEQ. ID NO: 19

Antibody

An antibody is a “Y”-shaped molecule and consists of two heavy chains and two light chains. There are different types and subtypes of both, the heavy chains and the light chains. Each heavy chain and each light chain has a constant region and a variable region, whereby the size of constant regions is bigger for the heavy chain.

The term “constant region” or “C region” refers to a region of an antibody molecule that is nearly identical with the corresponding regions of antibodies of different specificities produced by organisms of the same species. The constant part is invariable within the same antibody class (isotype) and is responsible for the effector functions of a particular Immunoglobulin subclass. An Fc fragment is a fragment of an antibody generated by the cleavage of an antibody with the enzyme papain and comprising the most part of the constant regions.

The term “variable region” as used herein refers to a region of an antibody molecule which binds to specific antigens. It is composed of the combination of the antigen-binding sites of the heavy and of the light chain. It differs between immunoglobulins of different B cells, but is the same for all immunoglobulins produced by the same B cell. The variable region is generated somatically via a gene recombination process taking place during maturation of B-cells. It is this process of rearrangement that creates the enormous diversity needed to bind any given antigen, and that thus enables the immune system to recognize and neutralize the innumerable antigenic burdens posed by foreign and pathogenic structures. As a consequence, the antibody pool is composed of immunoglobulins bearing a large repertoire of different V regions, while sharing the same Fc portions.

The term “fragment binding antigen” or “Fab fragment” is an antibody fragment that includes the variable, antigen-binding region, and which is generated by the cleavage of an antibody with the enzyme papain.

The term “idiotypic region” as used herein refers to the part of the variable region of an antibody that is unique for each antibody type.

The third transgenic nucleotide sequence encodes the light chain of antibody directed against the beta-amyloid peptide. Preferably, said antibody is a human, humanized or chimeric antibody. More preferably, said antibody is an immunoglobulin gamma (IgG). Most preferably, said antibody is the therapeutic antibody Mab11 (WO 03/070760).

The forth transgenic nucleotide sequence encodes the heavy chain of an antibody directed against the beta-amyloid peptide. Preferably, said antibody is a human, humanized or chimeric antibody. More preferably, said antibody is an immunoglobulin gamma (IgG). Most preferably, said antibody is the therapeutic antibody Mab11 (WO 03/070760).

The term “beta-amyloid peptide” or “A-beta” refers to a peptide produced by cleavage of APP by beta-secretase and gamma-secretase. The peptide has 39-43 amino acids. It is the main constituent of amyloid plaques in the brains of Alzheimer's disease patients. Preferably, the beta-amyloid peptide is a human beta-amyloid peptide.

Preferably, the forth transgenic nucleotide sequence encode for the heavy chain of a secreted antibody. Such a preferred DNA sequence lack sequences encoding a transmembrane domain.

The third and forth transgenic DNA sequences may be operably linked with one promoter (i.e. with the third promoter) or they may be operably linked each with a separate promoter (i.e. the third transgenic DNA sequence is operably linked with the third promoter and the forth DNA sequence is operably linked with the forth promoter).

Promoters

The term “operably linked” as used herein means that a DNA sequence and a regulatory sequence are connected in a way as to permit gene expression when the appropriate transcriptional activator proteins are bound to the regulatory sequence.

The term “regulatory sequence” as used herein refers to is a nucleotide sequence which is a binding site for proteins controlling the protein expression. A regulatory sequence may for example a promoter or an enhancer.

The term “promoter” or as used herein refers region of gene that binds RNA polymerase and transcription factors to initiate transcription.

The term “enhancer” as used herein refers to a nucleotide sequence that increases the rate of genetic transcription by increasing the activity of the nearest promoter on the same DNA molecule

The first promoter may be any promoter which expresses the operably linked DNA in the neurons. The first promoter may be an ubiquitary promoter. Preferably, the first promoter is a neuronal promoter. More preferably, the first promoter is a brain-specific promoter.

The term “tissue-specific promoter” refers to a promoter, which controls and directs expression of a gene in a tissue-specific manner, e.g., in brain tissue, in muscle tissue, in liver tissue, in kidney tissue, etc. A brain-specific promoter is a promoter which controls ad directs expression of a gene in brain tissue. Most preferably, first promoter is a promoter of a prion gene.

The first promoter may also be a controllable promoter. A controllable promoter may be any promoter, which controls the expression of a transgene in a regulatable and/or inducible fashion, e.g., by addition of specific inducer or repressor substances. Several inducible bacterial promoter are known in the art (Schultze N, Burki Y, Lang Y, Certa U, Bluethmann H; Nat Biotechnol 1996; 14(4): 499-503; van der Neut R; Targeted gene disruption: applications in neurobiology; J Neurosci Methods 1997; 71(1): 19-27; Liu H S, Lee C H, Lee C F, Su I J, Chang T Y; Lac/Tet dual-inducible system functions in mammalian cell lines. Biotechniques. 1998; 24(4): 624-8, 630-2).

The second promoter may be any promoter which expresses the operably linked DNA in the neurons. The first promoter may be an ubiquitary promoter. Preferably, the second promoter is a neuronal promoter. More preferably, the second promoter is a brain-specific promoter.

The term “tissue-specific promoter” refers to a promoter, which controls and directs expression of a gene in a tissue-specific manner, e.g., in brain tissue, in muscle tissue, in liver tissue, in kidney tissue, etc. A brain-specific promoter is a promoter which controls ad directs expression of a gene in brain tissue. Most preferably, the second promoter is a Thy1.2 glycoprotein promoter.

The second promoter may also be a controllable promoter. A controllable promoter may be any promoter, which controls the expression of a transgene in a regulatable and/or inducible fashion, e.g., by addition of specific inducer or repressor substances. Several inducible bacterial promoter are known in the art (Schultze N, Burki Y, Lang Y, Certa U, Bluethmann H; Nat Biotechnol 1996; 14(4): 499-503; van der Neut R; Targeted gene disruption: applications in neurobiology; J Neurosci Methods 1997; 71(1): 19-27; Liu H S, Lee C H, Lee C F, Su I J, Chang T Y; Lac/Tet dual-inducible system functions in mammalian cell lines. Biotechniques. 1998; 24(4): 624-8, 630-2).

The third promoter may an ubiquitary promoter or a lymphoid-tissue specific promoter.

Preferably, the third promoter is a promoter of the MHC class I gene. Also preferably, the third transgenic DNA sequence is operably linked with an enhancer. More preferably, the enhancer is an IgH enhancer.

The third promoter may also be a controllable promoter. A controllable promoter may be any promoter, which controls the expression of a transgene in a regulatable and/or inducible fashion, e.g., by addition of specific inducer or repressor substances. Several inducible bacterial promoter are known in the art (Schultze N, Burki Y, Lang Y, Certa U, Bluethmann H; Nat Biotechnol 1996; 14(4): 499-503; van der Neut R; Targeted gene disruption: applications in neurobiology; J Neurosci Methods 1997; 71(1): 19-27; Liu H S, Lee C H, Lee C F, Su I J, Chang T Y; Lac/Tet dual-inducible system functions in mammalian cell lines. Biotechniques. 1998; 24(4): 624-8, 630-2).

The forth promoter may an ubiquitary promoter or a lymphoid-tissue specific promoter. Preferably, the forth promoter is a promoter of the MHC class I gene. Also preferably, the fourth transgenic DNA sequences is operably coupled with an enhancer. More preferably, the enhancer is an IgH enhancer.

The fourth promoter may also be a controllable promoter. A controllable promoter may be any promoter, which controls the expression of a transgene in a regulatable and/or inducible fashion, e.g., by addition of specific inducer or repressor substances. Several inducible bacterial promoter are known in the art (Schultze N, Burki Y, Lang Y, Certa U, Bluethmann H; Nat Biotechnol 1996; 14(4): 499-503; van der Neut R; Targeted gene disruption: applications in neurobiology; J Neurosci Methods 1997; 71(1): 19-27; Liu H S, Lee C H, Lee C F, Su I J, Chang T Y; Lac/Tet dual-inducible system functions in mammalian cell lines. Biotechniques. 1998; 24(4): 624-8, 630-2).

As the transgenic non-human animal produces also its own antibodies, the human heavy and light chains may be mixed up with the endogenous heavy and light chains to form a mixed endogenous/transgenic antibody. In case of a mouse, mixed mouse/human antibodies may be formed. If the human heavy and light chains are produce outside the lymphoid tissue, no endogenous heavy and light chains will be present and therefore pure transgenic antibodies will be formed. Therefore, the promoter(s) operably linked to the third and fourth transgenic DNA sequence is preferably a promoter expressed in both, lymphoid and non-lymphoid tissue.

Transgenic Animal/Integration of the Transgenes

The transgenic DNA sequences are integrated in all or a portion of the animals cells, especially in the germ cells. The integration into the genome may be transient or stable. Preferably, the integration is stable.

The transgenic animal may be heterozygous or homozygous for the first, second, third and forth transgenic DNA sequences. The transgenic animal may be heterozygous for some of the transgenic DNA sequences, for example for the first and second transgenic DNA sequences, and homozygous for the other, for example for the third and fourth DNA sequences. Preferably, the transgenic animal is homozygous for at least one of the transgenic DNA sequences.

The transgenic animal may be any non-human animal. Preferably, the non-human transgenic animal is a mammal, more preferably a rodent such as rat or a mouse, most preferably, the non-human transgenic animal is a mouse.

Production of Transgenic Animals

The transgenic non-human animal may be any non-human animal. The preferred non-human animal is a mammal. More preferably, the non-human animal is a rodent such as a mouse or a rat. Methods for producing a transgenic non-human animal are well known in the art.

Suitable methods are described i.e. in Hogan B., Beddington R., Costantini F. & Lacy E. Manipulating the mouse embryo. A laboratory manual. 2nd Edition (1994). Cold Spring Harbor Laboratory Press.

The transgenic proteins may be ubiquitary expressed. Preferably, the proteins encoded by the first and second transgenic DNA sequence are expressed in the neurons. More preferably, the proteins encoded by the first and second transgenic DNA sequence are expressed in brain-tissue. Preferably, the proteins encoded by the third and forth transgenic DNA sequences are expressed in lymphoid tissue. More preferably, the proteins encoded by the third and fourth transgenic DNA sequences are expressed in both, lymphoid and non-lymphoid tissue.

The transgenic non-human animals described above may be analyzed genetically, molecularly and behaviorally.

Use

The quadruple transgenic mouse as described above can be used as a model for treatment of the Alzheimer's disease or as model for preventing AD. It allows to investigate the effect of an human anti-Aβ antibody on the onset and process of Aβ amyloidosis in a murine AD model. Since the transgenic animal is immunotolerant for the transgenic human mAb11 antibody, the effect of chronic treatment with a therapeutic antibody such as for example Mab 11 can be determined. Also, the effect on soluble Aβ levels and the process and kinetics of vascular amyloidosis can be followed.

Furthermore, the quadruple transgenic non-human animal as described above is an interesting tool for determining the effectiveness and effect of a preventive treatment of Alzheimer's disease with a human antibody.

Having now generally described this invention, the same will become better understood by reference to the specific examples, which are included herein for purpose of illustration only and are not intended to be limiting unless otherwise specified, in connection with the following figures.

### FIGURES

FIG. 1 shows a schematic representation of the APP processing. A) Amyloid precursor protein. The cleavage site of the three secretases are indicated (α: α secretase, β: β secretase, γ: γ secretase).

FIG. 2 shows a schematic representation of the amyloid precursor protein (APP) processing. APP can undergo proteolytic processing via 2 pathways. Cleavage by α-secretase occurs within the A-beta domain and generates the large soluble N-terminal APPα and a non-amyloidogenic C-terminal fragment. Further proteolysis of this fragment by g-secretase generates the non-amyloidogenic peptide p3. Alternatively, cleavage of APP by β-secretase occurs at the beginning of the Aβ domain and generates a shorter soluble N-terminus, APPβ, as well as an amyloidogenic C-terminal fragment (C99). Further cleavage of this C-terminal fragment by γ-secretase generates Aβ. Cleavage by γ-secretase or multiple γ-secretases can result in C-terminal heterogeneity of Aβ to generate Aβ40 and Aβ42.

[symbol] Cell membrane; [symbol] APPβ, ▪ Aβ, ▪ CTF

FIG. 3 shows a schematic representation of the genetic constructs.

A) Construct comprising DNA encoding human betaAPP751 with K60N, M671L substitution (human βAPP751-SFAD) embedded in the mouse Thy 1.2 glycoprotein gene (MThy1.2). KPI: Kunitz protease inhibitor domain; Aβ: beta amyloid peptide; EI: Exons I; EII: Exon II; EIV: Exon IV; ATG: start codon; TAG: stop codon.

B) Construct comprising the DNA encoding a mutant human presenilin 2 with N141 substitution (hPS2-N141I) embedded in the mouse prion (pPrPHG). E1: Exon 1, E2: Exon 2, UTR: untranslational region; ATG: start codon; TAG: stop codon.

C) Construct comprising DNA encoding the heavy chain of the human antibody IgG 1.1 (Mab11) directed against the beta-amyloid peptide. MHC-I: H-2k promoter region, VH: Variable regions of the heavy chain of the antibody, CH1-CH2-CH3: Constant regions of the heavy chain of the antibody, IgH enhancer: transcriptional enhancer element of the murine IgH gene

D) construct comprising DNA encoding the light chain of the human antibody IgG 1.1 (Mab11) directed against the beta-amyloid peptide. MHC-I: H-2k promoter region, VL: Variable regions of the light chain of the antibody, CL: Constant regions of the light chain of the antibody, IgH enhancer: transcriptional enhancer element of the murine IgH gene

FIG. 4 shows the entire coding sequence (SEQ. ID NO: 10) of the human Ig γ1 gene specific for human Aβ as cloned into the expression vector pHSE3′ for transgenic expression. The position and the name of the primers used in PCR amplification are displayed above or below the corresponding sequence. The Stop codon is shown in bold. Leader sequence is shown bold and underlined.

FIG. 5 shows the entire coding sequence (SEQ. ID NO: 11) of the human Ig κ gene specific for human Aβ as cloned into the expression vector pHSE3′ for transgenic expression. The position and the name of the primers used in PCR amplification are displayed above or below the corresponding sequence. The start and stop codon are shown in bold.

FIG. 6 shows a picture of the PCR electrophoresis gel to detect the quadruple transgenic in progeny of B6.Mab11xB6.PS2APP mice. Lane 1: marker, lane 2 to 7: progeny mice of B6.Mab11xB6.PS2APP mice. IgH: IgH specific PCR fragment; IgL: IgL specific PCR fragment. PSEN2: PS(N41I) specific PCR fragment.

FIG. 7 shows a Western Blot of brain homogenate from B6.MAB11, B6.152 and B6.MAB11.152 mice probed for APP.

B6.MAB11: mouse transgenic for the heavy and light chain of MAB 11. B6.152: mouse transgenic for PS2 (N141I)/APPswedish. B6.MAB11.152: mouse transgenic for the heavy and light chain of MAB 11 and for PS2 (N141I)/APPswedish. APP: Amyloid precursor protein, CTF: C-terminal fragment, Abeta: Beta amyloid peptide.

FIG. 8 shows fluorescence microscopy images of tissue section of the frontal cortex of wildtype, Mab11 transgenic mouse (B6.MAB11), PS2(N141I)/APPsw double transgenic mouse (B6.152) and the quadruple mouse transgenic for the heavy and light chain of MAB 11 and for PS2 (N141I)/APPswedish (B6.MAB11.152). The mice were 8 month old and the sections were stained with a murine anti-Ab monoclonal antibody (BAP2/Alexa 555 conjugate). The transgenic expression of MAB11 in the quadruple transgenic mouse completely prevents plaque formation.

FIG. 9 shows fluorescent microscopy images of tissue section of the frontal cortex of wildtype, Mab11 transgenic mouse (B6.MAB11), PS2(N141I)/APPsw double transgenic mouse (B6.152) and the quadruple mouse transgenic for the heavy and light chain of MAB 11 and for PS2 (N141I)/APPswedish (B6.MAB11.152). The mice were 8 month old and the sections were stained with thioflavin-S. The expression of MAB11 in the quadruple transgenic mouse completely prevents plaque formation.

FIG. 10 shows a graphical representation of measured levels of human IgG, i.e. MAB11 in plasma withdrawn from the Mab11 transgenic mouse (A) and the quadruple transgenic mouse (B6.MAB 11.152) (B). Different independent methods were compared, (i) human IgG captured by Aβ antigen and detected by an Fc specific detection antibody [symbol] , (ii) human IgG captured by anti-human IgG (H+L) and detected by an (H+L) specific detection antibody [symbol] and (iii) human IgG captured by anti-human IgG (H+L) and detected by an Fc specific detection antibody

[symbol]

FIG. 11 shows a Western Blot of brain homogenate from quadruple transgenic mouse B6.MAB11.152, the Mab11 transgenic mouse B6.MAB11 on a non-transgenic background (C57B1/6) probed with for human IgG Mab11. For determining the measurable minimal amount of Mab11, the antibody was added at different concentrations (10, 3, 1, 0.3, 0.1 and 0.03 μg/ml) to the brain homogenate of the non-transgenic mouse B6 (C57B1/6). Plasma withdrawn from the non-transgenic mouse is the control for Mab11 in the periphery.

FIG. 12 shows a western blot of brain homogenates from 16 month old mice probed for Aβ. The brains were taken from non-transgenic wildtype C57B1/6 mice, of quadruple transgenic mice B6.MAB11.152 and PS2(N141I)/APPsw double transgenic mouse B6.152. In the first lane MAB11 was added to sample buffer without brain homogenate as negative control.

FIG. 13 shows a graphical representation of measured unsoluble Abeta (Abeta40 and 42) after Aβ dissociation in the brain homogenate from B6.152 and B6.MAB11.152 mice (in ng/mg brain).

1-4: PS2(N141I)/APPsw double transgenic mouse (B6.152): 1=4 months old; 2=8 months old, 3=12 months old, 4=16 months old;
5-8: quadruple transgenic mouse (B6.MAB11.152): 5=4 months old; 6=8 months old, 7=12 months old, 8=16 months old.
A): Abeta 42 measured in females mice
B) Abeta 42 measured in male mice
C) Abeta 40 measured in female mice,
D) Abeta 40 measured in male mice.

FIG. 14 shows confocal scanning microscopy images visualized as maximal intensity projection of cortex sections taken from B6.MAB11.152 double-immunostained for vascular amyloid deposits and endothelial cells. FIGS. 14A and D show BAP2 staining of amyloid deposition, staining at low and high magnification, respectively. For comparison, FIGS. 14B and E show the distribution of capillaries by CD31 (endothelial cell marker) staining at low and high magnification, respectively. FIGS. 14C and F show the merged channels of both stainings.

FIG. 15 shows confocal scanning microscopy images visualized as maximal intensity projection taken from the double transgenic B6.152 mouse (A to C) and single confocal plane images of a cortical capillary from a quadruple transgenic B6.MAB11.152 (D to F) at high resolution. FIGS. 15A and D show BAP2 staining of amyloid deposits. FIGS. 14B and E show CD31 (endothelial cell marker) staining. FIGS. 14C and F show the merged channels of both stainings.

### EXAMPLES

Commercially available reagents referred to in the examples were used according to manufacturer's instructions unless otherwise indicated.

### Example 1

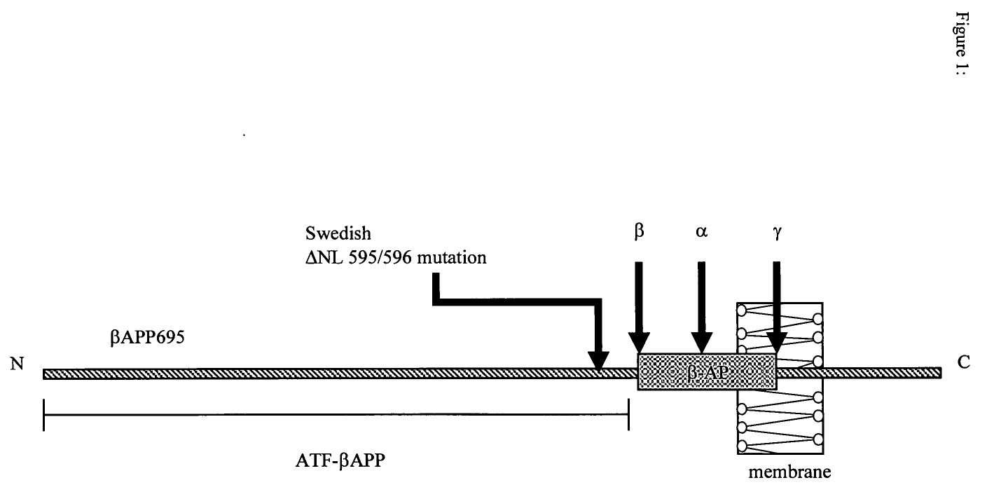

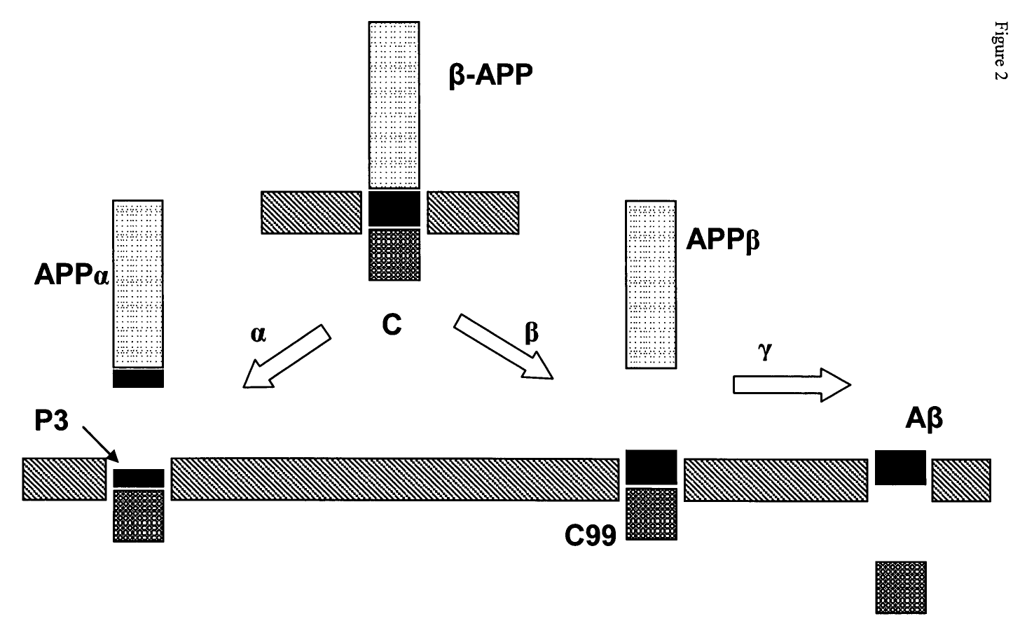

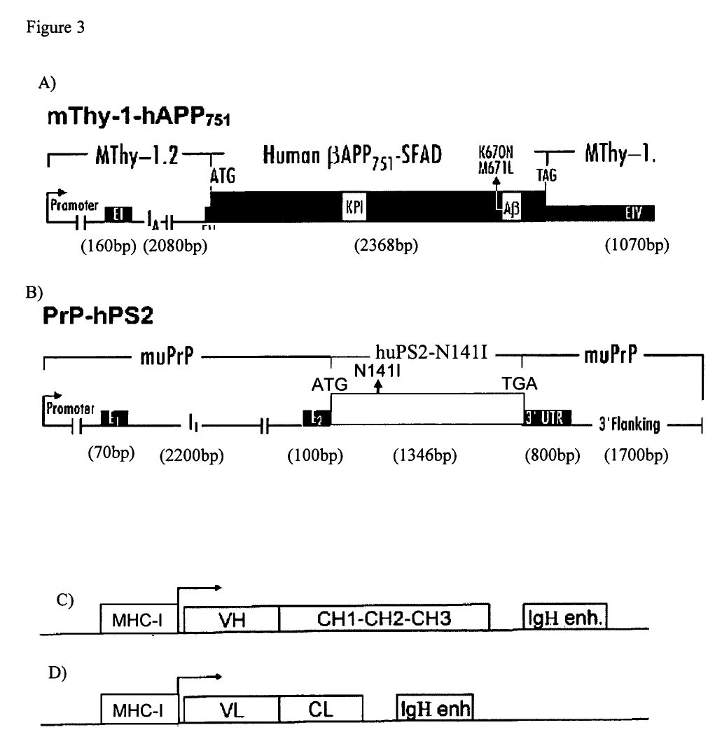

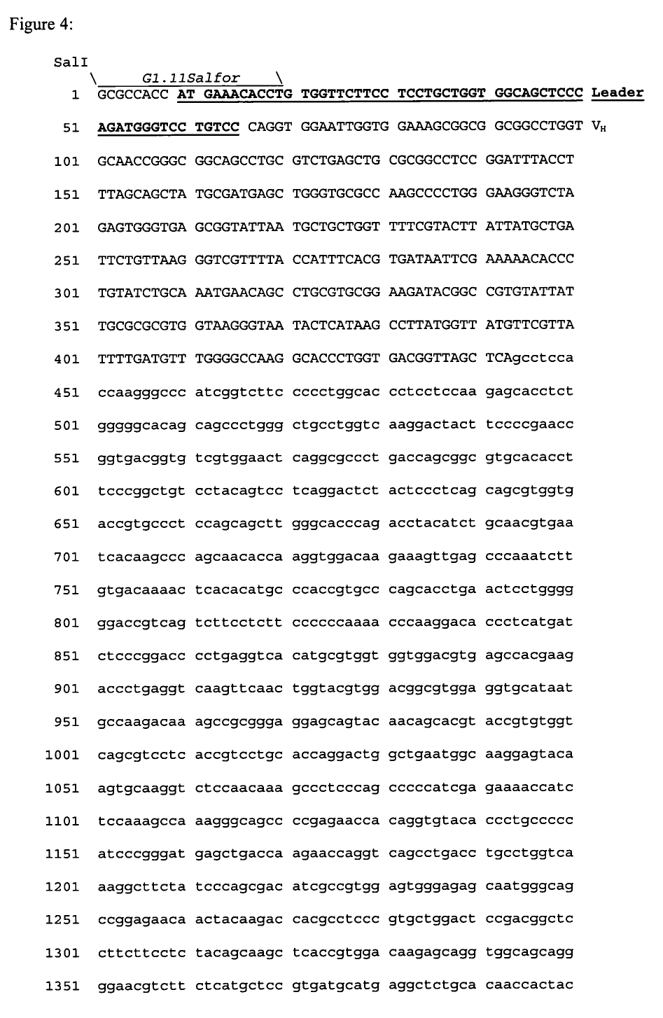

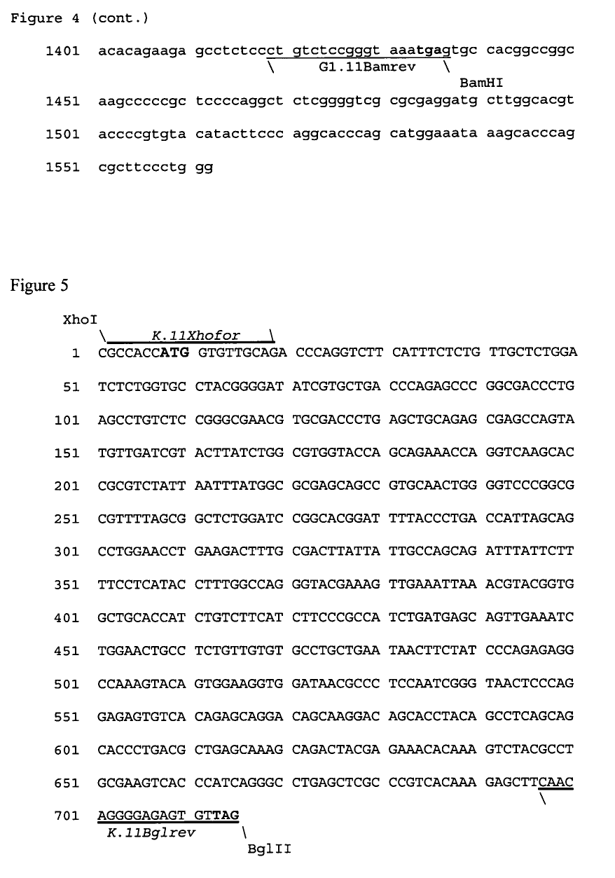

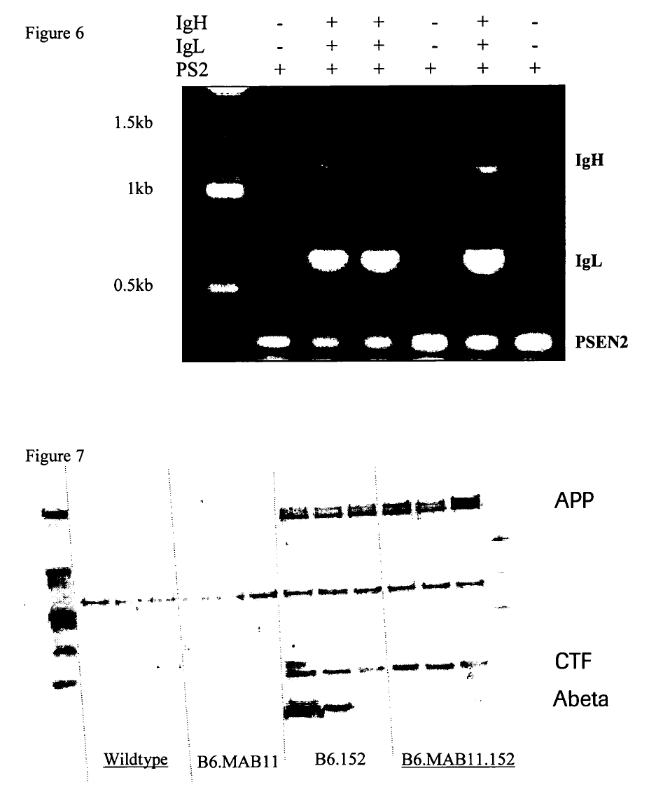

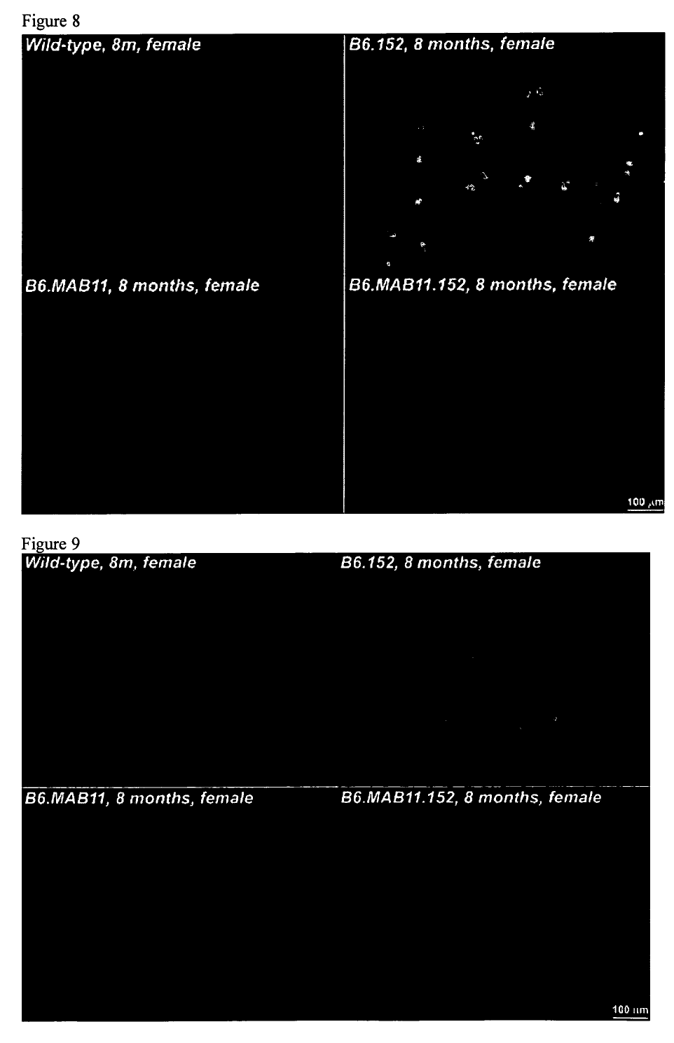

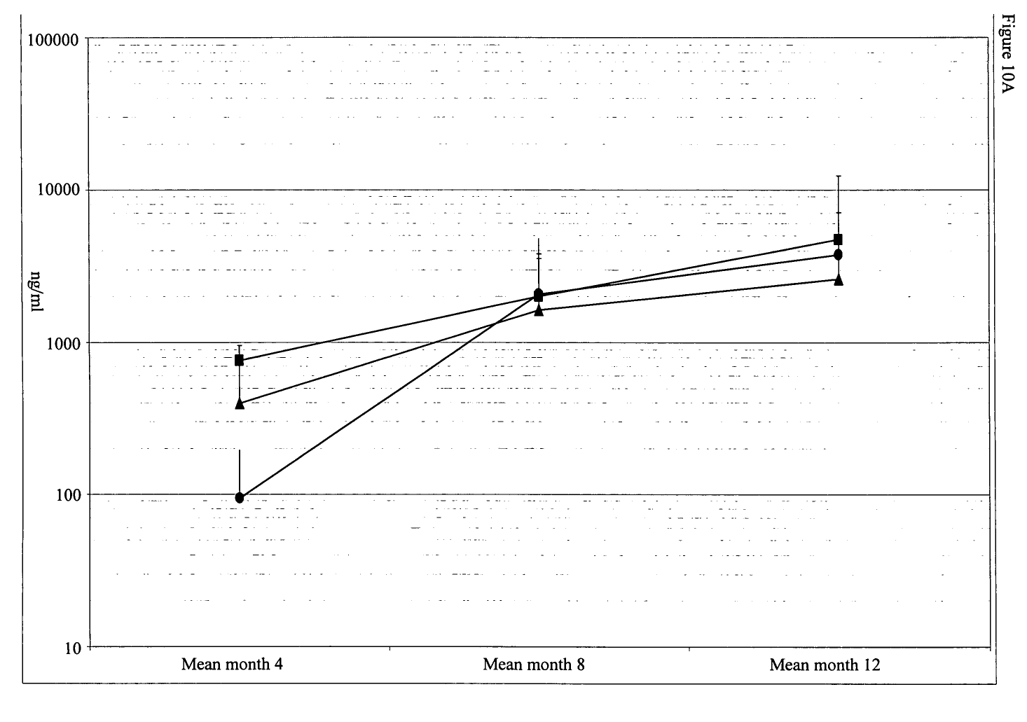

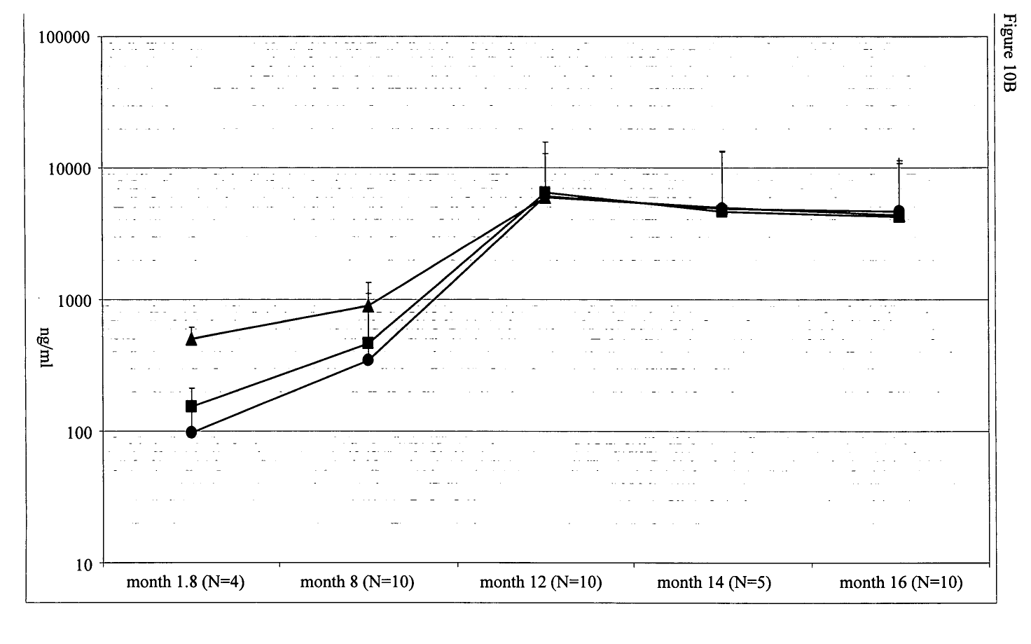

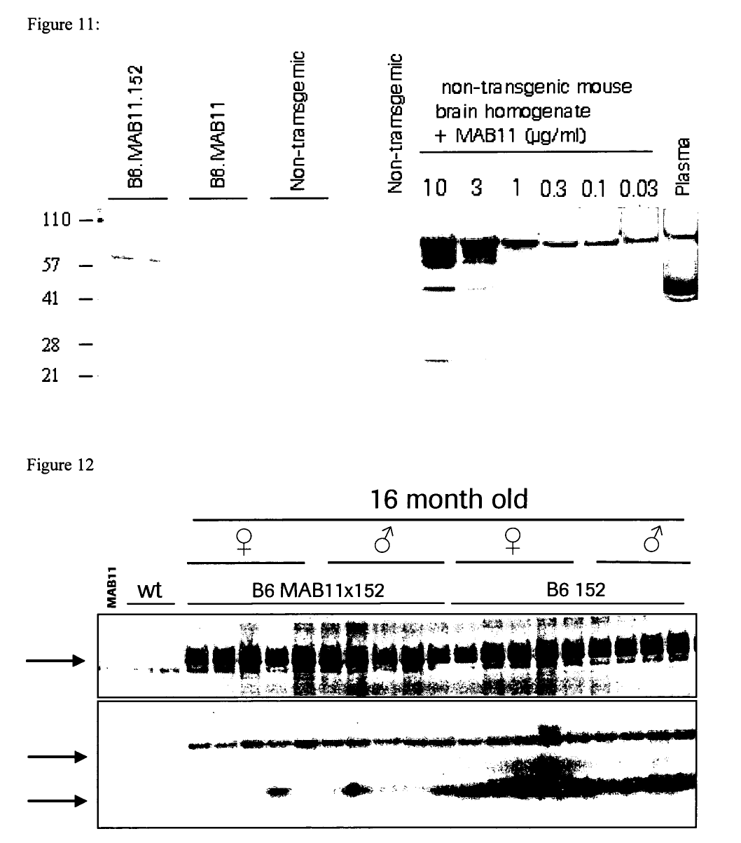

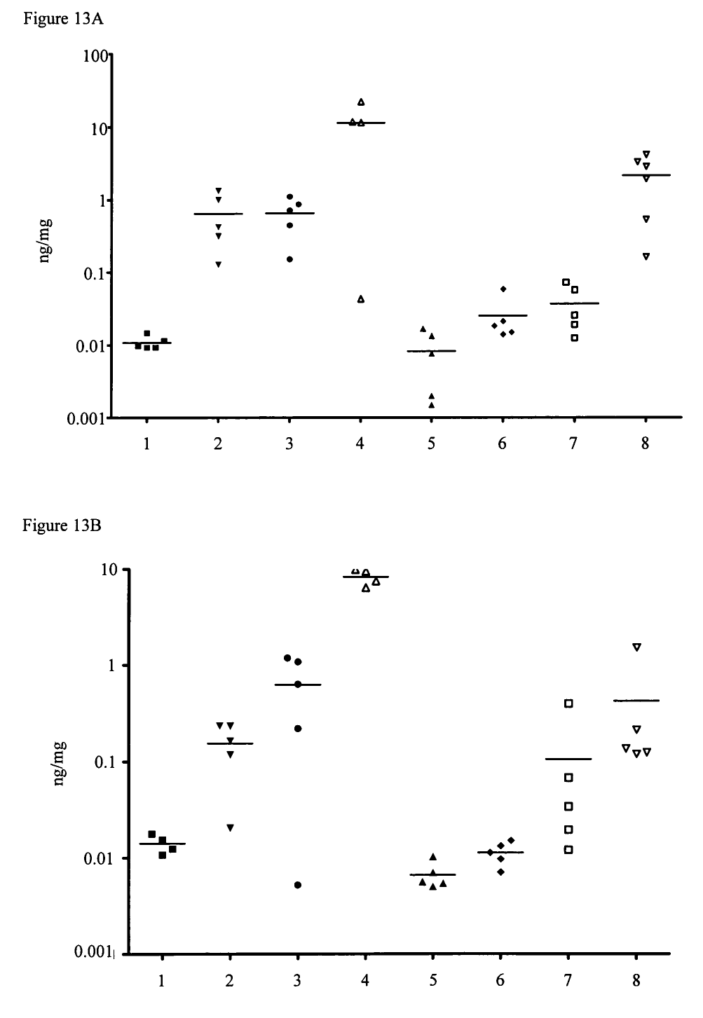

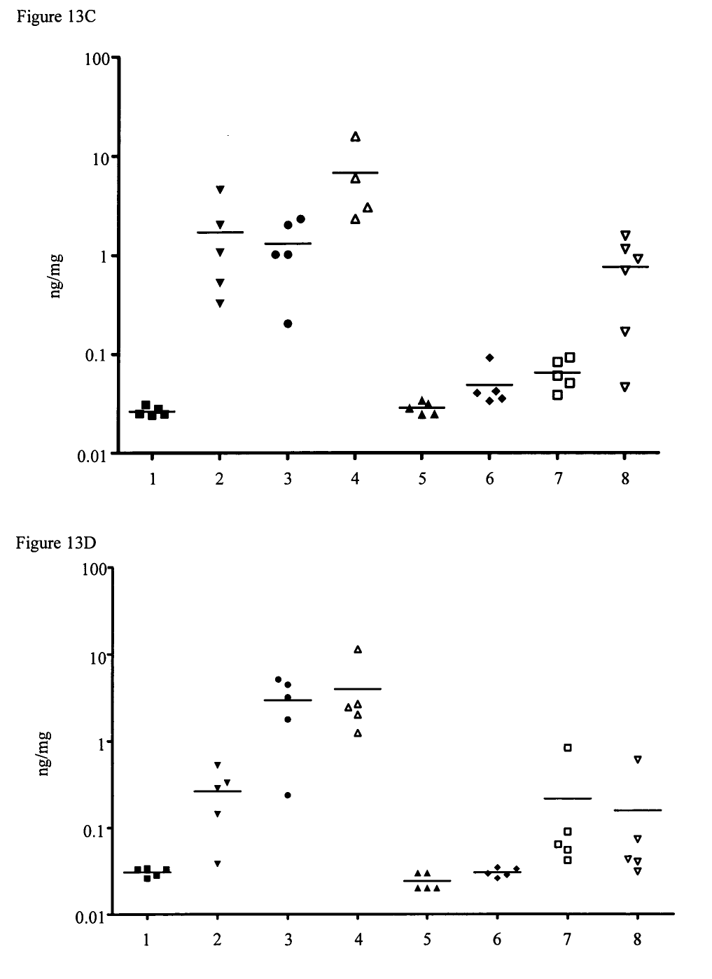

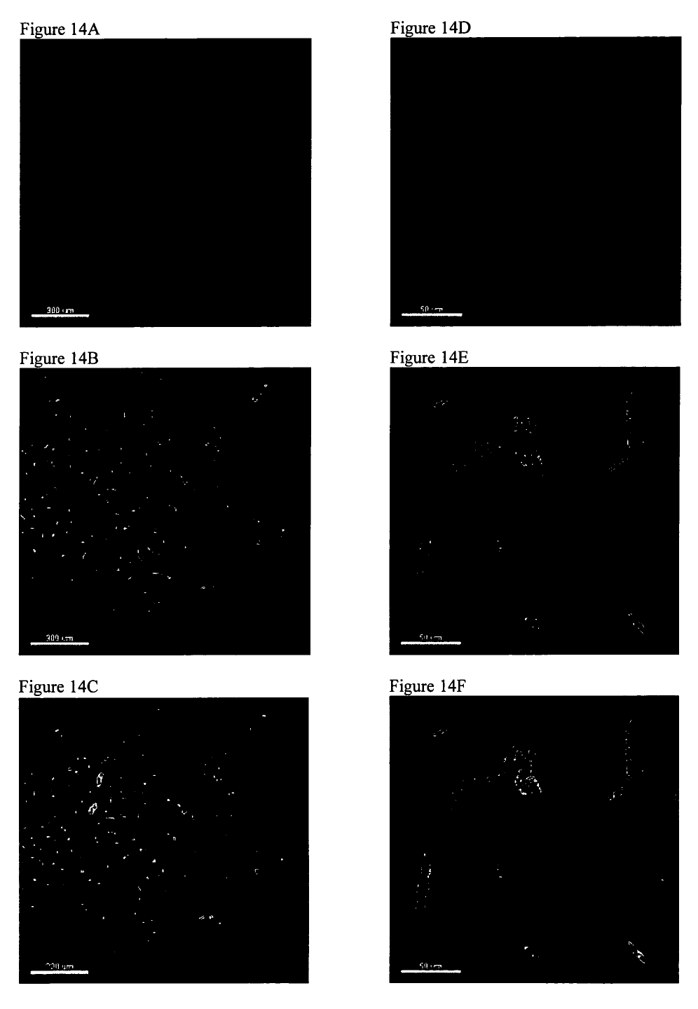

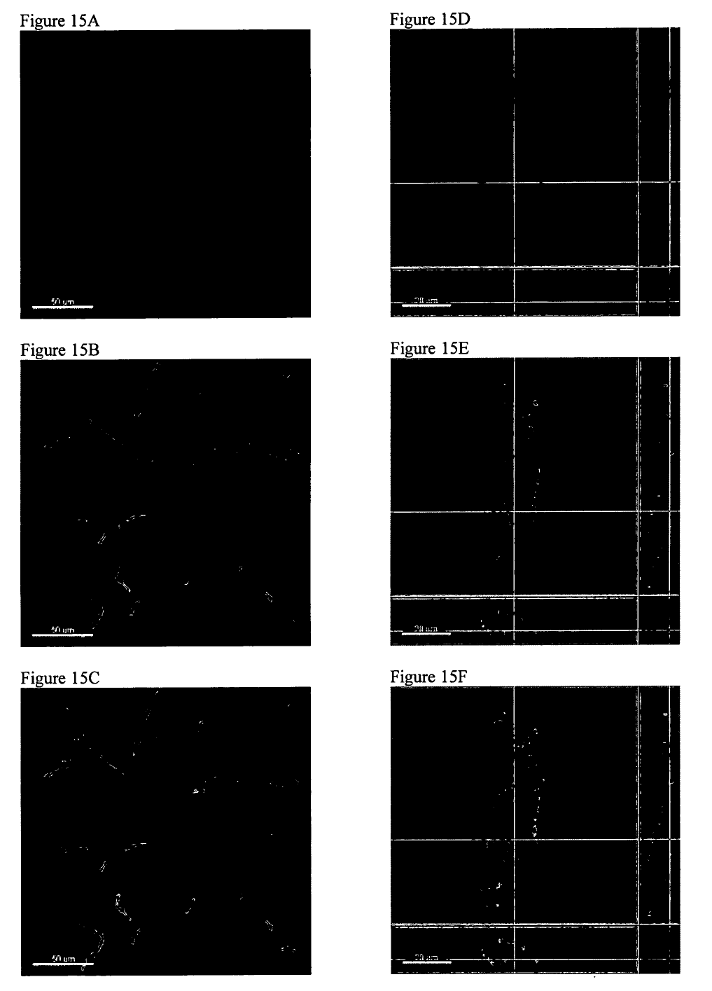

### Transgenic Constructs (FIG. 1)

APPSwe construct: The cDNA encoding human βAPP751 was donated by Prof. D. Goldgaber. The Swedish familial AD double mutation (SFAD, K670N, M671L substitution) was introduced by site-directed mutagenesis. The Thy1.2-huβAPP751SFAD transgene was generated by insertion of the approximately 2.4 kbp huβAPP751 SFAD cDNA fragment (SEQ. ID NO: 1: nucleotides 32-2369 of AC: X06989) into the XhoI site of an expression vector containing mouse Thy-1.2 glycoprotein gene and promoter (SEQ. ID NO:2, AC:M12379). This expression vector has a 6.8 kbp NotI fragment comprising mouse Thy1.2 gene (Andra K, et al., (1996) Expression of APP in transgenic mice: a comparison of neuron-specific promoters. Neurobiol Aging 17:183-190; Vidal M, et al., (1990) Tissue-specific control elements of the Thy-1 gene. EMBO J 9:833-840) in which the 1.5 kbp BamI-XhoI fragment located on exons 2 and 4 was replaced by the XhoI cloning site. Partial sequence of the construct is given in SEQ. ID NO: 4 (Nucleotides 1-2602, MMTHYGP, AC:M12379 (as of 27.4.1993), mouse Thy-1.2 glycoprotein gene (XbaI-BanI), Nucleotides 2603-4982, hβAPP751 SFAD (SmaI/HindIII-Blunted, 2400 bp), AC:X06989 (as of 09.01.2003), (nt:32-2400) with K670N, M671L, Nucleotides 4983-6112, MMTHYGP, AC:M12379 (as of 27.4.1993), XhoI-PolyA site (exon IV)

PSEN2mut construct: The FAD mutation N141I was introduced into a human PSEN2 cDNA (SEQ. ID NO: 9, AC: L43964). For expression in transgenic mice, a vector based on the mouse prion gene was used (pPrPHG) (Fischer et al., 1996; Borchelt et al., 1996). A KpnI-NarI fragment, encompassing the prion gene coding region, was deleted and replaced by a unique SceI site. The presenilin 2 coding sequence was inserted by blunt-end ligation and verified by sequencing of the insertion sites.

Mab11 construct: A cDNA encoding an immunoglobulin (Ig) heavy chain (H) of the isotype γ1 (SEQ. ID. NO: 10) and a cDNA encoding a light chain (L) of the isotype κ (SEQ. ID. NO: 11) and with specificity for human Ab peptide were used [Bardroff, M. et. al., Anti-amyloid beta antibodies and their use. WO03070760]. The cDNAs were amplified in a PCR reaction using the primers in Table 1. The 5′ primers contain a SalI (or compatible XhoI) site and the 5′ primers contain a BamHI (or compatible BglII) site for directed insertion into the pHSE3′ vector [Pircher, H., et al., T cell tolerance to Mlsa encoded antigens in T cell receptor V beta 8.1 chain transgenic mice. Embo J, 1989. 8(3): p. 719-27.]. The PCR-amplified cDNAs were first enzymatically cut with both restriction enzymes SalI and BamHI, and then individually inserted into the corresponding sites of the vector pHSE3′. The expression of the Ig cDNAs in pHSE3′ is driven by the murine promoter of the MHC class I gene H-2k and enhanced by the murine Ig H gene enhancer located 3′ of the cloned genes [Pircher, H., et al., T cell tolerance to Mlsa encoded antigens in T cell receptor V beta 8.1 chain transgenic mice. Embo J, 1989. 8(3): p. 719-27.]. This expression vector ensures high levels of production of the corresponding inserted gene products in T and B lymphocytes of the transgenic mice ([Pircher, H., et al., T cell tolerance to Mlsa encoded antigens in T cell receptor V beta 8.1 chain transgenic mice. Embo J, 1989. 8(3): p. 719-27.]). The entire expression cassette, including (from 5′ to 3′): the H-2k promoter, the inserted cDNA, the poly-A and splice sites and the Ig H gene enhancer element, was then excised from the vector by means of restriction enzyme cut with XhoI and agarose gel purification, and prepared in an adequate concentration for microinjection into fertilized mouse oocytes (2 μg/ml in 10 mM TrisHCl/0.1 mM EDTA, pH 7). The coding potential of the cDNA was confirmed by sequencing the entire cDNAs encoding the anti-Aβ Ig H and L genes.

TABLE 1
Sequences of cloning primers.

| | | Restriction site | SEQ. |
|---|---|---|---|
| Primer name | Sequence | (in bold) | ID No: |
| G1.11Salfor | 5′-ACGTGTCGACGCCGCCACCATGAAACACCTG-3′ | Sall | 12 |
| (5′IgH) | | (GTCGAC) | |
| G1.11Bamrev | 5′-ACGTGGATCCTCATTTACCCGGAGACAG-3′ | BamHI | |
| (3′IgH) | | (GGATCC) | 13 |
| K.11Xhofor | 5′-ACGTCTCGAGGCCGCCACCATGGTGTTGCAG-3′ | XhoI | 14 |
| (5′IgL) | | (CTCGAG) | |
| K.11Bglrev | 5′-ACGTAGATCTCTAACACTCTCCCCTGTTG-3′ | BglII | 15 |
| (3′IgL) | | (AGATCT) | |

### Example 2

### Generation of Transgenic Mice

Generation of B6.PS2APP transgenic mice (line B6.152): Transgenic animals were generated essentially as described previously (Hogan CF, and Lyacy E. 1995. Manipulating the mouse embryo: a laboratory manual. Ed 2. Plainview, N.Y.: Cold Spring Harbor Laboratory). Vector free linear fragments, carrying Thy-1-huAPP751SFAD or Prp-huPsen2(N141I) constructs were mixed in a 1:1 ratio and microinjected into male pronuclei of C57B1/6 zygotes. After microinjection, viable zygotes were implanted into the oviducts of pseudopregnant B6CBAF1 foster mothers. Integration of the transgenes in the germline was tested in the F1 progeny by detection of the transgenes by PCR. It is generally assumed that co-injected transgenes integrate into the same chromosomal site (Overbeek P A, Aguilar-Cordova E, Hanten G, Schaffner D L, Patel P, Lebowitz R M, and Lieberman M W. 1991. Coinjection strategy for visual identification of transgenic mice. Transgenic Res. 1, 31-37).

Generation of B6Mab11 (anti-Aβ IgG1 transgenic mice): The transgenic animals were generated as described previously in EP-A-1748294. Fertilized oocytes obtained from C57BL/6 female donors were microinjected with a 1:1 mixture of the purified XhoI fragments encoding for the IgH and L genes described in the previous section (Mab11 construct) to obtain the B6.Mab11 animals. After microinjection, viable zygotes were implanted into the oviducts of pseudopregnant B6CBAF1 foster mothers. Integration of the transgenes in the germline was tested in the F1 progeny by detection of the transgenes by PCR. It is generally assumed that co-injected transgenes integrate into the same chromosomal site (Overbeek P A, Aguilar-Cordova E, Hanten G, Schaffner D L, Patel P, Lebowitz R M, and Lieberman M W. 1991. Coinjection strategy for visual identification of transgenic mice. Transgenic Res. 1, 31-37). In total, 5 double transgenic mice bearing both the transgenic IgH and L genes and 1 single transgenic mouse bearing only the IgH gene were generated and bred.

Generation of B6.Mab11.PS2APP transgenic mice (Line B6.Mab11.152): Transgenic mice having both the transgenes for Mab11 and the human PSEN2mut and APPSw transgenes were generated by crossing males heterozygotes for the Mab11 transgenes with females homozygotes for huPSEN2mut and huAPPSw transgenes. The resulting progeny is either heterozygote for all transgenes or heterozygote for huAPPSw and huPSEN2 only.

### Example 3

### PCR Analysis

The presence of the transgenes is detected by PCR analysis using genomic DNA isolated from tissue biopsies taken on 10 days old mice. The fragments amplified are specific for the transgenes and do not cross-react with endogenous DNA sequences.

B6.PS2APP mice (APPswedish-PS2mut double transgenic mice): The primers 5′-AAG TAT GTC CGC GCA GAA CAG AAG G-3′ (SEQ. ID NO: 5) and 5′-CTG AGA TAT TTG AAG GAC TTG GGG AG-3′ (SEQ. ID NO: 6) are used to amplify a 932 bp fragment overlapping huAPP and muThy1 sequences. Similarly, the primers 5′-TCA TTG GCT TGT GTC TGA CCC T-3′ (SEQ. ID NO: 7) and 5′-GCT ITC AAC GTC AGT AGG ACA A-3′ (SEQ. ID NO: 8) are used to amplify a 327 bp fragment overlapping huPSEN2mut and prion sequences. The huPSEN2 and the huAPPSw transgenes always co-segregate. The PCR reaction was performed using 1 μl (about 100 ng) of total DNA obtained from toe clipping tissue, in PCR reaction with the following conditions: 3 min at 95°, 35× [30 sec at 95°, 1 min at 60°, 1 min at 72°], 5 min at 72°. The PCR-amplified DNA fragments of 932 by for the huAPP transgene and of 327 bp for the huPS2 transgene were visualized in 1.8% agarose gels.

B6.Mab11 mice (Mab11 double transgenic mice): Pups born from these microinjected embryos were screened for the presence of the transgenes by amplifying genomic DNA prepared from tail biopsies with specific primers. The primers used are indicated in Table 2. The PCR reaction was performed using 1 μl (about 100 ng) of total DNA obtained from the tail biopsy, in PCR reaction with the following conditions: 1 min at 90°, 30× [10 sec at 94°, 30 sec at 64°, 90 sec at 72°], 7 min at 72°. The PCR-amplified DNA fragments of about 1270 by and 660 by respectively were finally visualized in 1.5% agarose gels separately for the transgenic IgH and L genes.

TABLE 2
Primer sequences for detection of transgenes.

| PCR assay | Primers | | PCR fragment | SEQ. ID. |
|---|---|---|---|---|
| Ig H gene | 5H2KP | 5′-ATGAATTCACAGTTTCACTTCTGCACC-3′ | 1270 bp | 16 |
| | G1.0501 | 5′-TGTACTCCTTGCCATTCAGC-3′ | | 17 |
| Ig L gene | 5H2KP | 5′-ATGAATTCACAGTTTCACTTCTGCACC-3′ | 660 bp | 16 |
| | K.44 | 5′-GCTCATCAGATGGCGGGAAG-3′ | | 18 |

MAB11.PS2APP mice (FIG. 2): The primers 5′-ATG AAT TCA CAG TTT CAC TTC TGC ACC-3′ (SEQ. ID NO: 16) specific to the MHC promoter regions and the primers 5′-TGT ACT CCT TGC CAT TCA GC-3′ (SEQ. ID NO: 17) and 5′-GCT CAT CAG ATG GCG GGA AG-3′ (SEQ. ID NO: 18) specific to the gamma chain and kappa chain, respectively, are added to the huPSEN2 primers (SEQ. ID NO: 7 and 8) in a multiplex PCR reaction. In the presence of all transgenes, 3 PCR products of different sizes are amplified: 327 bp, 600 bp and of 1200 by for the huPSEN2, the hu Ig heavy chain and the hu Ig light chain transgenes, respectively.

The PCR reaction was performed using 1.5 μl (about 150 ng) of total DNA obtained from toe clipping tissue, in PCR reaction with the following conditions: 1 min at 95°, 30× [30 sec at 95°, 40 sec at 62°, 80 sec at 72], 7 min at 72°. PCR-amplified DNA fragments of 327 bp, of about 660 by and 1200 by were visualized in 1.5 agarose gels for the huPS2, IgH and L transgenes, respectively.

### Example 4

### Immunohistochemistry

Fluothane^{R}-anesthetised 8 months old mice (Wildtype mouse (C57B1/6), Mab11 transgenic mouse, PS2(N141I)/APPsw double transgenic mouse and PS2(N141I)/APPsw/Mab11 quadruple transgenic mouse) were decapitated brains halved and snap frozen in dry ice. Parasagittal cryostat sections between lateral ˜1.92 and 1.2 mm according to Paxinos and Franklin were cut at a nominal thickness of 20 micrometer at −18° C. using a Leica CM3050 S cryostat and mounted on Superfrost Plus™ slides and stored at −20° C. Staining of amyloid plaques was done by state of the art immunofluorescent staining protocols or staining with Thioflavin-S, a dye specific for amyloid plaques. Briefly, sections were hydrated in PBS and treated with 100% acetone precooled at −20° C. for 2 min. Washing with PBS was done twice for two minutes followed by blocking of unspecific binding sites by incubation in 2% bovine serum albumin and 2% ovalbumin in PBS for 15 min followed by an PBS wash and incubation with a high affinity murine monoclonal antibody (BAP-2), which was conjugated to Alexa488 fluorophore (Molecular Probes) w and incubated at 0.5 mg/ml for 1 hour at room temperature. Staining with Thioflavin-S was done at 0.0125% in ethanol/PBS (40/60 vol %). After rinsing the slides in bidistilled water glass coverslips were mounted using fluorescence mounting medium (S3023, DAKO). Results of 8 month old B6.MAB11.152 and B6.152 with littermate controls are shown in FIG. 8 (BAP-2) and FIG. 9 (Thioflavin-S) and demonstrate a complete absence of amyloid deposits in B6.MAB11.152 mice.

The tissue slides from cortex and near hippocampus from B6.MAB11.152 (20 month old) mice and B6.152 mice (20 month old) were prepared as described above for the 8 month old animals. The tissue slides were stained as follows:

1. rat/L anti-mouse CD31, 10.0 μg/ml (Serotec, ID: 1102) in PBS, pH 7.4+power Block (1×)+10% goat serum (pH7.4), 1 h incubation at room temperature, 3× rinsed with DakoCytomation Wash buffer (Tris buffer sodium chloride solution+Tween 20)

2. goat/anti-rat IgG(H+L) ALEXA 488, 1:100 (Molecular Probes, ID: 1254) in PBS, pH 7.4+10% goat serum (pH7.4), 1 h incubation at room temperature; 3× rinsed with DakoCytomation Wash buffer (Tris buffer sodium chloride solution+Tween 20)

3. mouse/BAP-2a ALEXA 555, 5.0 μg/ml (EM Labor, ID: 1296) in PBS, pH 7.4+power block (1×)+10% goat serum (pH7.4), 1 h incubation at room temperature; 3× rinsed with DakoCytomation Wash buffer (Tris buffer sodium chloride solution+Tween 20)

The slides were stained for 5 minutes with DAPI, then rinsed 2× with DakoCytomation Wash buffer, then incubate for 30 minutes with 4 mM CuS04.5H2O in 50 mM CH3COONH4 pH 5.0. After rinsing the slides in bidistilled water glass coverslips were mounted using fluorescence mounting medium (S3023, DAKO).

Results are shown in FIGS. 14 to 15. In the cortex of B6.MAB11.152 mice there are occassionally vascular amyloid depositions at thin capillaries. However, of B6.152 mice at the same age showed no vascular amyloid around cortical capillaries. At larger vessels, like the meningeal and ventricular vessels there was no obvious difference in the degree of vascular amyloidosis observed between B6.MAB11.152 and B6.152 mice.

### Example 5

### Western-Blot Analysis and Abeta Measurements

Western-Blot Analysis

15 μg of total protein extract was fractionated by SDS-PAGE and subsequently transferred onto a nitro-cellulose membrane (Amersham, Hybond). For Aβ detection, membranes were heated for 5 min at 95° C. in PBS (8.1 mM disodium hydrogen phosphate, 1.5 mM potassium dihydrogen phosphate, 137 mM sodium chloride, 2.7 mM potassium chloride) to increase epitope denaturation and presentation of epitopes, blocked with 5% (w/v) non-fat dry milk in PBST (PBS and 0.05% (v/v) Tween 20), and incubated overnight at 4° C. with the primary antibody. For detection of APP, the primary antibody W0-2 (Ida et al., J Biol Chem. 1996, 271(37):22908-1) was used at a concentration of 1 μg/ml. For the detection of presenilin-2 monoclonal antibody PST-20 detecting the amino-terminal fragment was used (Loetscher et al. J Biol Chem. 1997 Aug. 15; 272(33):20655-9), for tau antibody, Tau-5 (Invitrogen), for total APP antibody Anti-APP-CT20 (Calbiochem) and for human APP the antibody WO-2 which is specific for human APP (Ida et al., J Biol Chem. 1996, 271(37):22908-1). All washing steps were done with PBST at 4° C. Binding of the primary antibody was revealed with horseradish peroxidase conjugated anti-IgG antibody (Amersham) followed by ECL detection system according to the manufacturer's instructions (Amersham).

Abeta Measurements

One brain hemisphere was homogenized in 10 volumes (w/v) of 9M Urea, 50 mM Tris-HCl pH 7.4 containing a cocktail of protease inhibitors (Complete™, Roche Diagnostics GmbH, Mannheim-Germany). Cellular debris were removed by centrifugation at 4° C. for 5 min at 1500 g. The protein concentration in the supernatant was measured using the BCA protein Assay (Pierce, USA).

Aβ40 and Aβ42 levels in brain homogenates were determined using liquid phase electrochemiluminescence (LPECL) as described in Czech et al. (J Neurochem, 2007. 101(4): 929-36). Briefly, M-280 paramagnetic beads (Bioveris) were diluted with assay buffer (50 mM Tris, 60 mM NaCl, 0.5% BSA, 1% Tween 20, pH 7.4). 50 μl of brain extract was incubated at room temperature with 50 μl of beads and 25 μl of labeled antibodies (6E10-bio and BAP24-tag for detection of Aβ1-40, 6E10-bio and BAP24-TAG for detection of Aβ1-42) in a final volume of 250 μl for 3 h with gentle shaking. ECL was quantified using an M-Series M8 analyzer (Bioveris). Different dilutions of brain homogenates with assay buffer were measured to assure linear signal response and compared against synthetic standard Aβ1-40 and Aβ1-42 peptides (Bachem). Aβ peptides were solubilized in DMSO at a concentration of 1 mg/ml, aliquoted and stored frozen at −80° C. Results are expressed as ng/mg of brain homogenate referring to the standard curves of Aβ1-40 and Aβ1-42, respectively. Measurements were done in duplicate for each sample dilution and repeated at least two times.

### Example 6

### qRT-PCR of mab11 in B6.mab11.152 Mice

Spleenocytes were isolated from minced whole spleens by passing through a 70 μM cell strainer and serially diluted for RNS extraction. Brain samples were taken with laser microdissections from cortex, cerebellum and subiculum were provided collected into RNAlater®. RNA was extracted using the RNAeasy kit from Qiagen. PCR were performed with the QuantiTect Probe RT-PCR Kit (Qiagen). The TaqMan® Assay (Applied Biosystems Inc) was performed according to the instruction of the manufacturer. The probes were muIGH6_Mm01718956_m1 (IgM=B-cell marker), muAPP Mm00431827_m1 (reference gene for brain), huHC_custom (custom-made by ABI based on mab11 IgG1 const), and huLC_custom (custom-made by ABI based on mab11 IgKappa const).

The PCR was run on the LC480 (Roche Diagnostics). The relative quantitation of the target RNAs were performed by ΔCt method. Brain microdissections from non-transgenic mice gave no positive signal for the Mab11 IgH and IgK RNAs. Results for B6.mab11.152 mice are given in table 3 and 5.

TABLE 3
Expression of huIgH and huIgK in brain of B6.mab11.152 mice

| | ave ΔCp | SD | ΔΔ | % of APP |
|---|---|---|---|---|
| dIgHCX | 4.17 | 1.32 | 0.055 | 5.5 |
| dIgKCx | 2.41 | 0.58 | 0.188 | 18.8 |
| dIgHCb | 4.23 | 0.75 | 0.053 | 5.3 |
| dIgKCb | 2.93 | 0.69 | 0.131 | 13.1 |
| dIgHSub | 4.32 | 1.12 | 0.050 | 5.0 |
| dIgKSub | 2.71 | 1.32 | 0.153 | 15.3 |

The expression for endogenous reference genes IgM and CD20 in wildtype and transgenic mice was cell-number dependent. Only in the transgenic mice robust and cell-number dependent signal for huIg was found. In the wildtype mice no signal was detected vs. assay background (see table 4 and 5).

TABLE 4
Ct for B6.mab11.152 mice.

| cell no. | IGH6_55 | IGH6_56 | CD20 | huIgHG1 | huIgKC |
|---|---|---|---|---|---|
| 10000 | 22.2 | 25.0 | 24.9 | 29.0 | 24.4 |
| 1000 | 25.5 | 28.0 | 28.0 | 32.2 | 27.3 |
| 100 | 28.6 | 31.5 | 30.8 | 35.1 | 30.8 |
| 10 | 31.2 | 34.7 | 32.8 | 37.9 | 34.6 |
| 1 | 32.7 | 38.1 | 33.1 | | 38.0 |

IGH6_55, IGH6_56 = endogenous IgM reference,
CD20 = endogenous CD20 reference,
huIgHG1, huIgKC = Transgene-specific probes.

TABLE 5
Ct for wildtype C57B1/6.

| cell no. | IGH6_55 | IGH6_56 | CD20 | huIgHG1 | huIgKC |
|---|---|---|---|---|---|
| 10000 | 20.8 | 23.3 | 23.2 | 44.5 | 38.4 |
| 1000 | 24.0 | 26.4 | 26.4 | | |
| 100 | 27.2 | 29.7 | 29.6 | 38.5 | 36.3 |
| 10 | 30.1 | 33.5 | 32.2 | 38.1 | |
| 1 | 31.9 | 36.5 | 33.5 | | |

IGH6_55, IGH6_56 = endogenous IgM reference,
CD20 = endogenous CD20 reference,
huIgHG1, huIgKC = Transgene-specific probes.

1. A transgenic non-human animal whose genome comprises
a) a first transgenic DNA sequence encoding a human APP Swedish or a human APP London protein, wherein said first transgenic DNA sequence is operably linked to a first promoter;
b) a second transgenic DNA sequence encoding a human Presenilin 2 protein comprising a N1411 substitution, wherein said second transgenic DNA sequence is operably lined to a second promoter;
c) a third transgenic DNA sequence encoding a light chain of a antibody directed against the amyloid peptide, wherein said third transgenic DNA sequence is operably linked to a third promoter and;
d) a fourth transgenic DNA sequence encoding a heavy chain of said antibody, wherein said fourth transgenic is operably linked to the third promoter or to a fourth promoter.

2. The transgenic animal of claim 1, wherein the third transgenic DNA sequences encodes the light chain of anti-Abeta antibody Mab11, and wherein the fourth transgenic DNA sequence encodes the heavy chain of anti-Abeta antibody Mab11.

3. The transgenic animal of claim 2, wherein the first and second promoters are neuronal promoters.

4. The transgenic animal of claim 2, wherein first and second promoters are brain-specific promoters.

5. The transgenic animal of claim 4, wherein the third and/or fourth promoter is a promoter of the MHC class I gene.

6. The transgenic animal of claim 5 wherein the third and/or fourth promoter is combined with a IgH enhancer.

7. The transgenic animal of claim 6 wherein the animal is homozygous for at least one of the transgenic DNA sequences.

8. The transgenic animal of claim 7, wherein the animal is a non-human mammal.

9. The transgenic animal of claim 8, wherein the animal is a mouse.

10. A descendant of the non-human transgenic animal of claim 9.

11. A cell line or primary cell culture derived from a non-human transgenic animal of claim 9.

12. A tissue or an organ explant or culture thereof, derived from a non-human transgenic animal of claim 9.

13. (canceled)

14. A method for generating a transgenic non-human animal whose genome comprises transgenic DNA encoding a human APP Swedish or APP London, a human Presenilin 2 protein comprising N1411, substitution and a heavy and a light chain of a human antibody directed against the beta amyloid peptide comprising
a) co-injecting a genetic construct comprising a first transgenic DNA sequence encoding a human APP Swedish or APP London, and a genetic construct comprising a second transgenic DNA sequence encoding a human Presenilin 2 protein with a N1411 substitution into a non-human zygote or an non-human embryonic stem cell, and generating a double transgenic non-human animal from said zygote or embryonic stem cell;
b) co-injecting a genetic construct comprising a third transgenic DNA sequence encoding a light chain of a human antibody directed against the amyloid peptide, and a genetic construct comprising a fourth transgenic DNA sequence encoding a heavy chain of said human antibody into a non-human zygote or a non-human embryonic stem cell and generating a double transgenic non-human animal from said zygote or embryonic stem cell, and
c) mating transgenic animals of a) and b) and thereby producing a quadruple transgenic non-human animal whose genome comprises transgenic DNA encoding, a human APP Swedish or APP London, a human Presenilin 2 protein comprising a N1411 substitution, and a heavy and a light chain of a human antibody directed against the beta amyloid peptide.

15. A method for generating a transgenic non-human animal expressing a human APP Swedish or APP London, a human Presenilin 2 protein comprising N1411 substitution, and a heavy and a light chain of a human antibody directed against human beta amyloid peptide comprising
a) co-injecting a genetic construct comprising a first transgenic DNA sequence encoding a human APP Swedish or APP London wherein said first transgenic DNA sequence is operably linked to a first promoter, and a genetic construct comprising a second transgenic DNA sequence encoding a human Presenilin 2 protein with a N1411 substitution into a non-human zygote or an non-human embryonic stem cell, wherein said second transgenic DNA sequence is operably linked to a second promoter, and generating a double transgenic non-human animal from said zygote or embryonic stem cell;
b) co-injecting a genetic construct comprising a third transgenic DNA sequence encoding a light chain of a human antibody directed against the human amyloid peptide, wherein said third transgenic DNA sequence is operably linked to a third promoter and a genetic construct comprising a fourth transgenic DNA sequence encoding a heavy chain of said human antibody into a non-human zygote or a non-human embryonic stem cell, wherein said fourth transgenic DNA sequences is operably linked to a fourth promoter, and generating a double transgenic non-human animal from said zygote or embryonic stem cell, and
c) mating transgenic animals of a) and b) and thereby producing a quadruple transgenic non-human animal expressing a human APP Swedish or APP London, a human Presenilin 2 protein comprising a N1411 substitution, and a heavy and a light chain of a human antibody directed against the beta amyloid peptide.

16. The method of claim 15, wherein the first and second promoter are neuronal promoters.

17. The method of claim 15 wherein the first and second promoter are brain-specific promoters.

18. The method of claim 17, wherein the third and fourth transgenic DNA sequences encode the light and heavy chain of the anti-Abeta antibody Mab11.

19. The method of claim 18, wherein the first promoter is a Thy1.2 glycoprotein promoter.

20. The method of claim 19, wherein the second promoter is a prion gene.

21. The method of claim 20, wherein the third and/or fourth promoter is a promoter of the MHC class I gene.

22. A quadruple transgenic non-human animal produced by the method of claim 21.

23. (canceled)

24. (canceled)
